(19) United States
(12) Patent Application Publication
(10) Pub. No.: US 2023/0255549 A1
(43) Pub. Date: Aug. 17, 2023

(54) An Analytical Mode of Interdependent Movement Patterns of Upper and Lower Jaw Teeth by Bidirectional Synchronisation of Digital Image Acquisition of These Movements with Haptic Technology in Digital Analysis of Chewing, a Positioning Patterns and a Digital Chewing Recorder

(71) Applicant: SZCZERBANIEWICZ, Joanna, Czestochowa (PL)

(72) Inventor: Blazej SZCZERBANIEWICZ, Czestochowa (PL)

(21) Appl. No.: 18/017,393

(22) PCT Filed: Jul. 14, 2021
(86) PCT No.: PCT/PL2021/000047

§ 371 Date: Jan. 20, 2023

(30) Foreign Application Priority Data
Jul. 20, 2020 (PL) P.434728

(51) Int. Cl. A61B 5/00; A61B 5/11; G06T 7/246; G06T 7/292

(52) U.S. Cl. CPC A61B 5/4542; A61B 5/1127; G06T 7/246; G06T 7/292; G06T 2207/10021; G06T 2207/30036

(57) ABSTRACT

A mode of analysis of a pattern of interdependent movements of the upper jaw teeth and of the lower jaw teeth by bidirectional synchronising of the digital movement image acquisition technology with haptic technology in a digital analysis of chewing, on the feedback principle, using the Motion Capture technology, based on the identification of the position of preferably optical markers and recording their movements in camera systems, wherein the cameras to be preferably integrated into, at least, two systems, one on the right and the other on the left side of the mouth/face, or as one system to be centrally positioned, opposite to the centre line of the face, wherein each of the systems has at least one, preferably three, cameras including at least one and preferably two monochromatic or colour cameras of a minimum resolution of 2.3 Mpx.

An analytical mode of interdependent movement patterns of upper and lower jaw teeth by bidirectional synchronisation of digital image acquisition of these movements with haptic technology in digital analysis of chewing, a positioning pattern and a digital chewing recorder

The subject of the patent is an analytical mode of the pattern of interdependent movements of the upper and lower jaw teeth by bidirectional synchronising of the digital image acquisition technology of these movements with haptic technology, employing a positioning pattern with or without markers and a digital chewing recorder for the digital analysis of chewing in order to apply the results of the mode in question for the implementation of prosthetic and orthodontic embodiments, as well as, for example, in orthognathic surgery.

A mode of visualisation of human movements, known as Motion Capture, employs a camera or a set of cameras and markers, where the markers are stuck on the body surface in predefined anthropometric being spatial figures, stickers with a printed pattern, diodes, etc., the movement of which is recorded by the cameras. The reconstruction of the movements from the data, acquired by the camera from the markers, is carried out in a digital process on the computer image of the patient and uses the information about the movement of the markers. Using the above-mentioned mode certain information is obtained, concerning the skeleton structure and the range of motion, where the joints constitute the articulation points, which are the pivot points, while the bone/skeleton is a stationary point of reference with regards to which the movements of the studied object are mapped by means of the minimisation of the distance between the markers and the reference point determining marker (the method of least squares, optimal superposition-Prokrust's method or recording in the conditions of existent unknown appropriateness—the Iterative Closest Point—ICP), that makes it possible to reconstruct movement. The use of Motion Capture finds applications in medicine, where it is broadly used in the physiotherapy of motor organs, including the masticatory organ. The above-mentioned mode is therefore applied for the reconstruction of the position and movement of larger elements, e.g. in determining the parameters of limb vibrations and of the movement phases of the body parts that are at the highest risk of collapse.

A measurement device and a method of teeth position recording have been demonstrated on Polish Patent No. P.231343, the essence of which is the method by which the measuring devices are fixed on the patient's head and record the anatomical reference points of the head and the jaws, as well as determine the positions of particular teeth in space, using measurement devices, markers, cameras, etc., placed along the conventional symmetry axis of the patient's face. Using the solution in question, data are obtained, concerning the appearance and mutual position of elements, e.g. of teeth in space.

A measurement device and a method of teeth position recording have been demonstrated on Polish Patent No. P.231343, where the mandible movements are recorded in space, using the Motion Capture technology and the reference points, used to record the interdependent work of the upper jaw teeth and of the lower jaw/mandible teeth, and transfer its work to a digital environment.

A three-dimensional device, as demonstrated on Chinese patent No. CN10501238A, is used to trace the pathway of the mandibular movement. This device is used in a virtual articulator to record the activity of the lower jaw/mandible teeth and transfer the obtained data to a digital environment.

A method and system, as demonstrated on French patent No. FR2015/052816, are used to model the kinematics of the patient's mandible, using a system of 2 infrared cameras and a system of markers, fixed both to the skull, provisionally to the upper teeth and, during the entire measurement cycle, to the lower teeth of the patient, as well as to the indicator, allowing for a stereoscopic reconstruction of the positions of the teeth relative to one another, and of the patient's face relative to selected characteristic measurement points, and the movements of the teeth of the lower jaw relative to the upper jaw in real time and to record the movements in the computer memory for further analysis and data processing, using the models of teeth of the upper and lower jaws, acquired earlier by the 3D scanning technique.

In the above-mentioned solutions, the natural/artificial teeth of the upper jaw and the natural/artificial teeth of the lower jaw are approached as rigid 3D objects of lasting/unchangeable shapes, not undergoing any displacements in the bone of the alveolar process of the jaw or the mandible, thus not taking into account the, so-called, alveolodental ligament (attachment apparatus) (periodontium) or of the implant/bone junction as a result of interdependent/mutual collisions, occurring in the course of the execution of movements, carried out by the 3D objects/upper/lower teeth, especially of chewing and thus do not provide for the mutual work of the teeth relative to the periodontium or the implant/bone junction at the moment of collision of the teeth crowns during the work of both jaws. In other words, they do not consider the mutual displacement of teeth at collision points/in the course of collisions, thus the changes of their position relative to the other teeth, which are invisible to the naked eye within the range from 0.2 to 1 mm, while being significant during the work of the masticatory organ, should be considered during the construction prosthetic embodiments or in the course of subsequent stages of orthodontic treatment.

Haptic technology is known, using mechanic communications of devices/robots, by means of virtual environment/software, with users/people by the tactile sense, making use of varying forces, vibrations or movements, recorded by haptic devices/haptic manipulators (HM). Its task is to release a tactile stimulus, signalling an interaction between the robot and the man. The record of 3D object dislocations, obtained by the method of digital image correlation, may be the source of data for this type of simulation. It creates the possibility to convert the digitally acquired image data (in which it concentrates mainly on the reading of the displacement of the markers, stuck on 3D objects, in the function of time) into data, read out by encoders, mainly optical, which then, by means of suitable sensors (cylinders) and software, process the data by special control algorithms and generate control signals to the actuating system of the haptic robot, i.e., its motors. An input signal, e.g. the effector's position, is transferred to the control unit, which, by means of appropriate algorithms, converts these signals and then, on their basis, generates output signals. The output signals (control by selected motors in the haptic robot) are properly amplified and then are sent to the actuating system, i.e. to the motors in the haptic manipulator (HM). The driving systems of the haptic devices then generate an appropriate torque and, in effect, act on the operator with the correct force (e.g. a variable in the form of vibrations), that allows for the evaluation of the correlation degree between the course of vibration amplitude size at the time of contact/collision for the two shapes, generated by dislocations of a certain amplitude in a predefined time unit and the course of the amplitude height of these dislocations. In order for the haptic robot to naturally impacted our tactile sense, the cycle of information acquisition and transfer, the so-called haptic loop, should be carried out with the frequency of 1 kHz. The haptic technology is also broadly implemented in medical fields, especially in all types of simulators. The use of vibration mechanisms achieve particularly realistic sensations, which are identical to those that will be experienced, for example, in a future surgeon during procedure. Today's machines will ideally simulate the resistance of a tissue when a probe is attempted to be inserted or the force that will be needed to make a precise cut. (https://spidersweb.pl/2015/03/haptic-feedback.html)

There is a known technology of individually designed orthodontic appliances (braces), used for the transfer of jaw scans and castings to the digital environment in which orthodontic appliances are individually designed. Using this method, an individual system of locks is designed, tailored to a given patient, which are then installed separately on each tooth. (https://ortodonta.com/ipa-pl/)

There is a known technology of stitching 3D objects, acquired in CAD technology or by 3D scanning, with the pictures of these objects, recorded by the video film technique, thanks to the optical/reflexive markers mounted on 3D objects, followed by stitching the 3D objects with markers, either scanned or created in a CAD/CAM environment, with the images of the same 3D objects with the same markers, recorded by the video technique (stitching of 3D objects, acquired from CAD/CAM or 3D scan, into the video film, thanks to the optical markers). (https://www.gom.com/metrology-systems/aramis/aramis-3d-camera.html)

The aim of the patent is to describe the mode of analysis of the pattern of interdependent movements of the natural/artificial teeth of the upper jaw and of the natural/artificial teeth of the lower jaw by bidirectional synchronising of the movement image acquisition technology with haptic technology, using a positioning pattern and a digital recorder used in the digital analysis of chewing by the application of scanning technology and/or recording with a camera/system of cameras, preferably of optical or infrared markers (indirectly, in the external part of the positioning pattern—M1 and/or directly on the surface of the veneered crowns of teeth—M2), preferably optical or retroflexive, recording the displacements of particular natural/artificial teeth or of the supra-gingival structure, seated on implants, relative to themselves and/or to the bone of the dental process of the upper/lower jaw and of the face in a video film, and a feedback connection with the haptic manipulator (HM).

A significant feature of the mode of analysis of the interdependent movement pattern of the teeth of the upper jaw and of the teeth of the lower jaw by bidirectional synchronising the digital movement image acquisition technology with haptic technology on the feedback principle in the digital analysis of chewing is the use of Motion Capture technology, based on the identification of the positions of markers, preferably optical, and on the systems of cameras, recording the marker movements, where the cameras are arranged in at least two systems, one on the right and the other on the left side of mouth/face, or in one system, centrally positioned opposite to the centre line of the face, whereas each of the systems possesses at least one, preferably three, cameras, including, at least one (stereoscopic) and two monochromatic or colour cameras of a minimum resolution of 2.3 Mpx, and with a high frame time rate of a minimum of 1,000 frames per second (FPS), with a dedicated optics/lens (preferably with the focus from 35 mm and a diaphragm from f 1.4), and, at least, one colour camera with a high resolution of a minimum of 12 Mpx and a low frame rate of a minimum of 25 FPS, enabling the recording of markers, fixed indirectly (M1) by means of the positioning pattern, or directly (independently—M2—preferably with the use of the individual design technology), to the veneer surface of the crowns of the natural/artificial teeth (placed on the natural teeth or seated on implants) in the upper jaw and, independently, to the veneer surface of the crowns of the natural/artificial teeth (placed on the natural teeth or seated on implants) in the lower jaw (M1 and/or M2), whereas the operation of the cameras is synchronised in time, thanks to their connection with triggering cards, and is responsible for recording of the position/movement of the crowns of the upper teeth (natural/artificial on implants), with M1 and/or M2 markers relative to the crowns of the lower teeth (natural/artificial teeth on implants), with M1 and/or M2 markers and of the crowns of the above-mentioned teeth (natural/artificial on implants), upper and/or lower, relative to the bone of the dental recess of both jaws and relative to the patient's face with M2 markers, stuck on it in characteristic anatomical points (preferably a minimum of 3 markers at the region of the temporomandibular joints, on the right and left side, and on the agger nasi), thanks to the simultaneous recording in the video film technology, and then (thanks to M1 and/or M2 markers) to stitching of the 3D scans of the upper and lower teeth with M1 and/or M2 into video film, where the scans are acquired by the intraoral or extraoral scanning technique, and enables the conversion of the movement of M1 and/or M2 markers, recorded by the system of cameras during movement in the course of the patient's chewing analysis, and transferred to the software environment, where the digital acquisition of the data from M1 and/or M2 markers is submitted to further analysis, enabling the conversion of the movements of these markers into the number of collisions between the crowns of the upper and lower teeth (natural/artificial on implants) with M1 and/or M2 markers fixed to them, which are preferably read by haptic manipulators, allowing for the mapping/control of these collision-caused relocations in the form of vibrations by the tactile sense of the frequency between 1 and 4 kHz, thus allowing the rating of the course of amplitude changes in the provisional angle of collision, lower/higher than 10 degrees between the pairs of the crowns of teeth (natural/artificial on implants), upper and lower, with M1 and/or M2 markers, in a set unit of time, divided into time intervals, preferably 0.001-second intervals, on the pathway of each movement phase/chewing cycle, i.e. on the pathway of adduction to intercuspation, and of abduction from intercuspation (on average, on the pathway of 0.2 mm with a time of 116 ms—for intercuspation, on average, on the distance from 1.3 mm to 1.5 mm during 200 ms for the adduction phase to intercuspation and the abduction phase from intercuspation), expressed by the number of vibrations on the haptic manipulator at the moment of contact/collision of these pairs of crowns of the upper and lower teeth (natural/artificial on implants) with M1 and/or M2 markers in a predefined unit of time.

A significant feature, according to this invention, is that the positioning of the location of the implants, embedded into the bones of the upper and/or the lower jaw (in case of a total anodontia of the upper and lower jaw) is carried out by means of the supragingival structures, seated on the implants (for example, impression connectors/transfers/transfers for scanning by the 3D technique—scan post/scan base) by making for them and rigid fixing on them of individually designed para-occlusion brackets/spoons, constituting the internal part of the positioning pattern, connected with the ready-made, prefabricated external part of this pattern, protruding from the mouth and tipped with at least three markers, preferably stickers, in order to designate the spatial setting of the positioning pattern and, in this way, of the supragingival structures, seated on the implants, relative to the system of recording cameras, what allows for the positioning of the teeth/supragingival structures, seated on the implants, relative to one another within one dental arch, relative to the teeth/supragingival structures, seated on the implants of the opposite dental arch, and for the entrance into the patient's face with M2 markers, stuck on it at characteristic anatomical points (min. 3 markers at the region of the temporomandibular joints on the right and left side and on the agger nasi) and/or a video film.

A significant feature according to the invention is that in case of the lack of space in dental occlusion or in articulation movements of the crowns of the upper/lower teeth relative to each other, resulting in an inability to record free movement, a method of the markers positioning is used (also for orthodontic treatment procedures) by a direct plotting/positioning of the markers (M2) on the veneer surface of the crowns of the teeth (natural/artificial on implants), preferably on the crown of each tooth separately, preferably by the use of the method of individually designed orthodontic appliances.

A significant feature of the pattern, positioning the crown of the teeth, (natural/artificial on implants), is that it consists of two separable parts, i.e. the external, ready-made, prefabricated part, protruding from the mouth and tipped with at least three markers, preferably optical, so as to designate the spatial setting of the positioning pattern with the markers (M1) relative to the recording cameras, and of the internal part, individually designed and tailored to the veneer surface of the crowns of the patient's teeth (natural/artificial on implants), whereas the internal part and the external part are rigidly joined with each other.

A significant feature of the digital chewing recorder is that its monolithic enclosure possesses a recess geometry, such as an arch, made in such a way as to simultaneously record—from either side, right and left—the three-dimensional shape of the face (concave, convex, flat), whereas on the ends of the arched recess there are two sets of cameras (or one set, centrally positioned opposite to the centre line of the face), recording independently or synchronically the mouth and either side of the patient's face and the visible markers, M1 and/or M2, joined with the crowns of the upper and lower teeth (natural/artificial on implants), whereas each of the camera sets includes three cameras at a predefined distances towards one another, whereas each set possesses two monochromatic or colour cameras of a minimum resolution of 2.3 Mps and a high frame rate of a minimum of 1,000 FPS, with a dedicated optics/lens (preferably with the focus from 35 mm and a diaphragm from f 1.4) and one colour camera with a high resolution of a minimum of 12 Mpx and a low frame rate of a minimum of 25 FPS, whereas the operation of both sets of cameras is synchronised by a triggering card(s) (preferably analogue-digital), acquiring information, and is responsible for recording the position/movement of the crowns of the upper teeth (natural/artificial on implants) with M1 and/or M2 markers relative to the crowns of the lower teeth (natural/artificial on implants) with M1 and/or M2 markers and relative to the patient's face with M2 markers, stuck on it at characteristic anatomical points (min. 3 markers at the region of the temporomandibular joints on the right and left side and on the agger nasi), thanks to their simultaneous recording on video film technology and then stitching (thanks to M1 and/or M2 markers) into the video film of 3D scans of the upper and lower teeth with M1 and/or M2 markers, acquired by the intra- or extraoral scanning technique, and enables the conversion of the dislocation of M1 and/or M2 markers, recorded by the system of cameras during movement in the course of examination, e.g. of the patient's chewing, and the transfer of the examination results to the software environment, where the digital acquisition of data from the optical markers, M1 and/or M2, undergoes further analysis, enabling the conversion of the markers dislocation into the number of collisions, which occur between the crowns of the upper and lower teeth (natural/artificial on implants) and the markers, M1 and/or M2, fixed to them, which are read out by haptic devices, preferably haptic manipulators, enabling the mapping/control of these dislocations, resulting from the collisions of the crowns of the teeth, such as vibrations by the tactile sense of the frequency between 1 kHz and 4 kHz.

The advantage of the solution in question is that additional information is obtained, concerning the mutual correlation of the teeth of the upper jaw and of the teeth of the lower jaw/mandible during activity, e.g. chewing, including the interdependence of the amplitude of dislocations of particular pairs of the crowns of natural/artificial teeth on implants at collision points, and their transfer to the digital environment by the reading of the information from the cameras and markers, enabling in this way the monitoring of the course of the amplitude changes in the provisional collision angle, smaller/larger than 10 degrees between the pairs of the crowns of the upper and lower teeth (natural/artificial on implants) with M1 and/or M2 markers, in a set time unit, divided into time intervals, preferably 0.001 second intervals, on the pathway of each phase of the movement/chewing cycle, i.e. in the way of adduction to intercuspation and in the way of abduction from intercuspation (on average, on 0.2 mm distance during 116 ms—for intercuspation, on average at a distance from 1.3 mm to 1.5 mm during 200 ms—for the phase of adduction to intercuspation and the phase of abduction from intercuspation, respectively), expressed by the number of vibrations on the haptic manipulator at the moment of contact/collision of these pairs of crowns of the upper and lower teeth (natural/artificial on implants) with M1 and/or M2 markers in a set unit of time.

Another advantage of the solution is that the obtained information can be transferred to the haptic manipulator, HM, in the form of vibrations, enabling an analysis/control by the tactile sense of the amplitude of dislocations of the occlusive surfaces of the crowns of teeth and/or of future prosthetic embodiments, made at the laboratory, participating in the earlier recorded movement patterns, including the patient's chewing movements, understood as the mobility of a tooth/supragingival structure, supported on an implant relative to the implant itself in the bone of the dental process of the upper and/or lower jaw, thanks to the periodontal fibres (no fibres in case of an implant), leading to the formation of a normal or disturbed, so-called functional envelope on the patient's own teeth, as well as on prosthetic embodiments, made at the laboratory and seated on the patient's own teeth or implants, and also in each of the phases of orthodontic treatment or after procedures in orthognathic surgery.

Another advantage of the solution is that the earlier recorded and defined vibrations from HM may reversely (by the feedback mechanism) determine/control/impose/prompt such and not another movement pattern of the upper teeth relative to the lower teeth, individually correlated to every patient thanks to the provisional angles of collision, smaller/larger than 10 degrees between the contact surfaces of the crowns of the teeth in a set unit of time, divided into time intervals, preferably 0.001 second intervals, with an amplitude not exceeding 0.2 mm in a set unit of time in each phase of the chewing cycle which, in case of construction of new prosthetic devices, orthodontic treatment and orthognathic procedures, will protect the periodontium from overload or the crowns on implants from damage. [By dedicated algorithms in the software thanks to the proper high-pass filtration for dislocation signals from the markers (recorded by the system of cameras) for all freedom degrees, allowing for a separation of the so called background movement, that is for removal of the constant component. i.e. the common, variable phase of the chewing movement for the basic dislocations, common for all the movements/collision of teeth, in which the dislocation distance of a tooth in the periodontium is below 0.2 mm in a set unit of time (on average 200 ms), divided into time intervals, preferably 0.001 second intervals, therefore, for the constant (variable) component of the chewing movement, the number of collisions in a set unit of time (on average 200 ms) is constant and does not cause any deformation of the periodontium ligaments, which would exceed 0.2 mm, therefore, it will not be accompanied by any vibration.]

Another advantage of this solution is a possibility to measure the range of teeth mobility, exerted by a stomatological activity (prosthetic, orthodontic, orthognathic) thanks to the recording of the markers dislocation on the crowns of the upper and lower teeth (natural/artificial on implants), reflecting/imitating the movements of the periodontium of these teeth from 0.1 to 1.0 mm (preferably above 0.2 mm to 1.0 mm), resulting from the varying amplitude of the provisional collision angle, smaller/larger than 10 degrees between the contact surfaces of these crowns in a set unit of time, divided into time intervals, preferably 0.001 second intervals, on the pathway of each movement phase/chewing cycle, i.e. on the pathway of adduction to intercuspation and of abduction from intercuspation, causing, in particular, a deformation of the periodontium, exceeding 0.2 mm, as an example of disturbed functional work of the teeth relative to one another, i.e. a disturbed functional envelope in result of the above-mentioned stomatological intervention.

Another advantage is the determination of the position and movement of M1 and/or M2 markers, while monitored by the system of cameras, unlike in other patents (where the placement of markers on the upper and lower teeth serves the positioning of 3D scans of the teeth relative to one another but not to the face, where the scans are obtained by the internal or external scanning technique, and serves the monitoring of the movements of the lower teeth relative to the upper teeth and not of the upper/lower teeth/crowns on implants relative to one another and to the bone of the dental process in which they are anchored), thanks to the stitching of M1 and/or M2 markers from 3D scans of the upper and/or lower teeth/crowns on implants with the images of Ma and/or M2 markers on the crowns of the upper and/or lower teeth/crowns on implants from the video film, being 0.1 mm, preferably 0.05 mm.

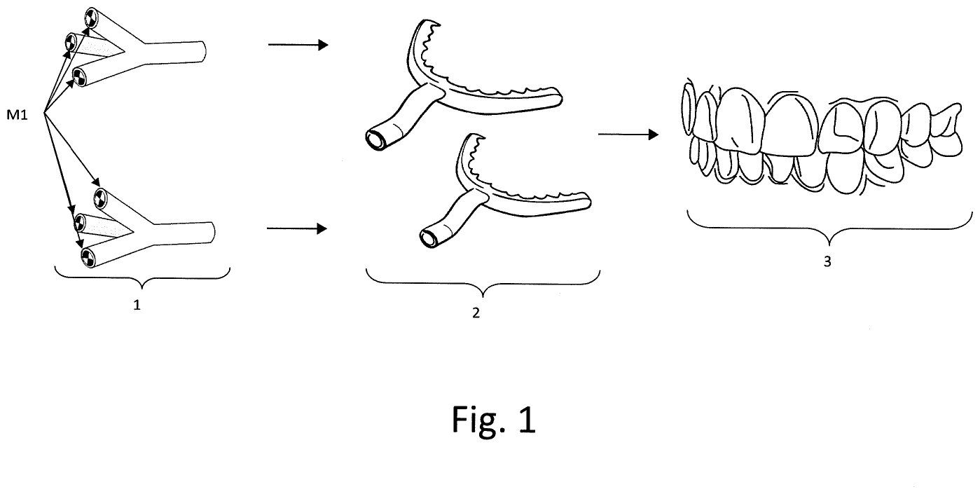

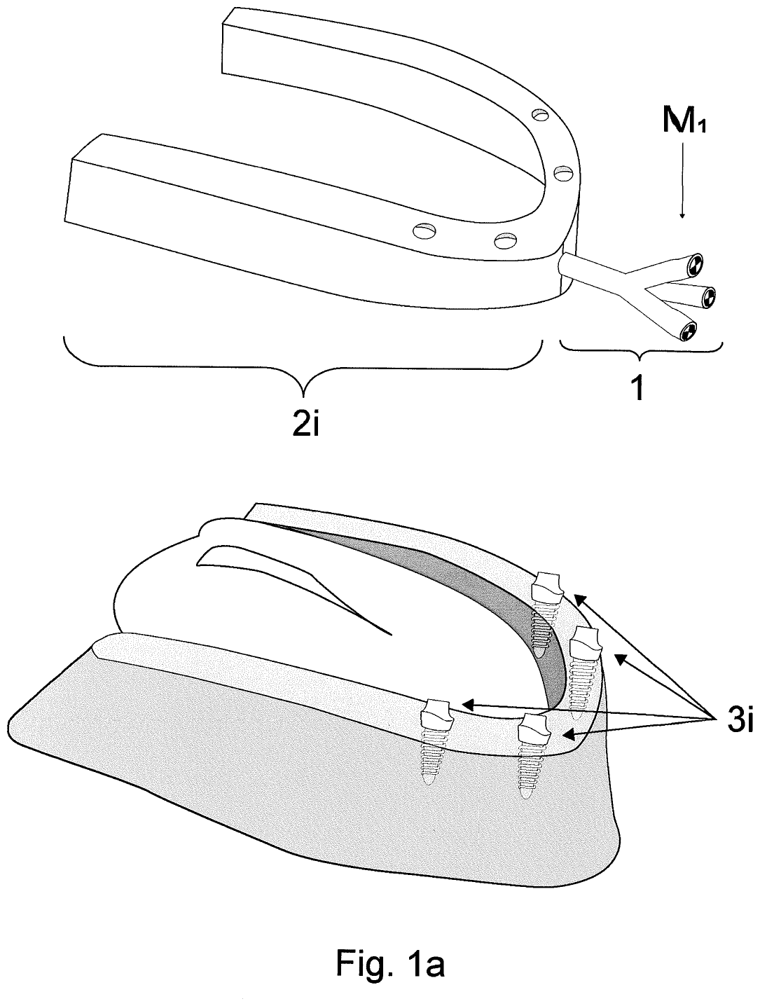

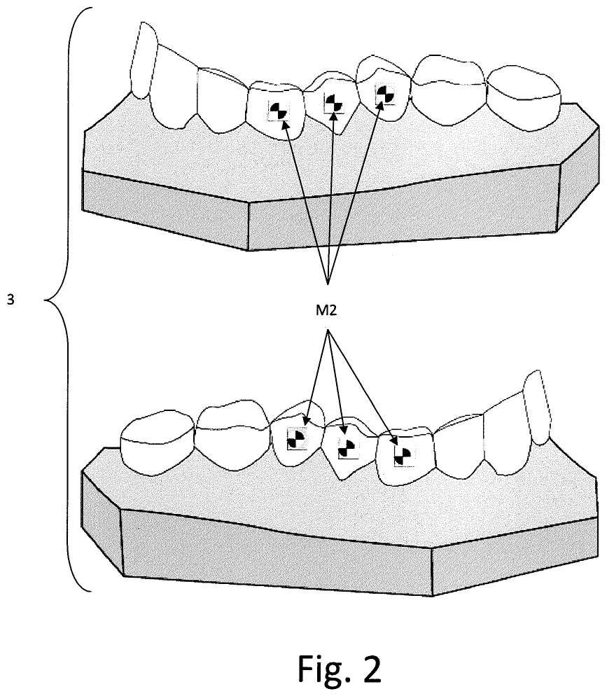

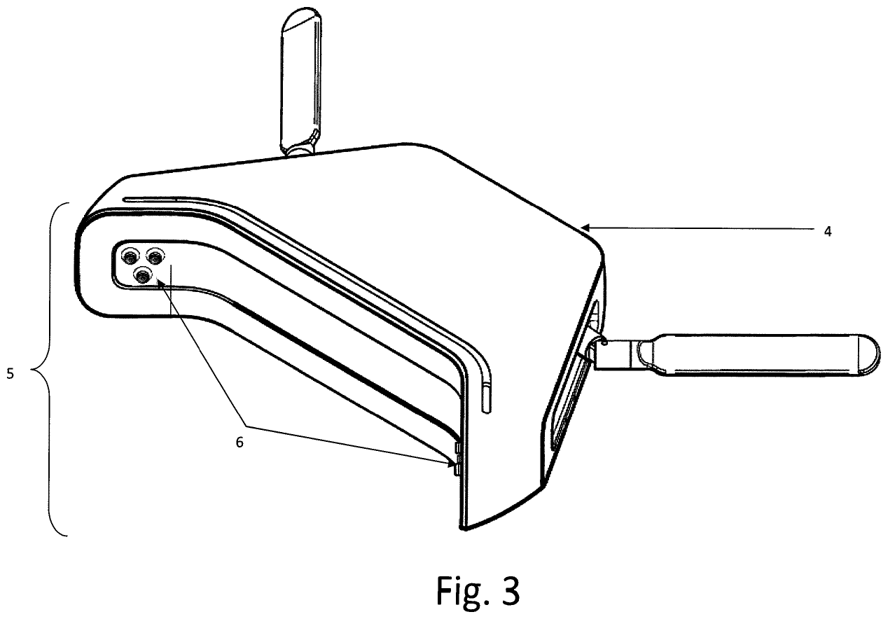

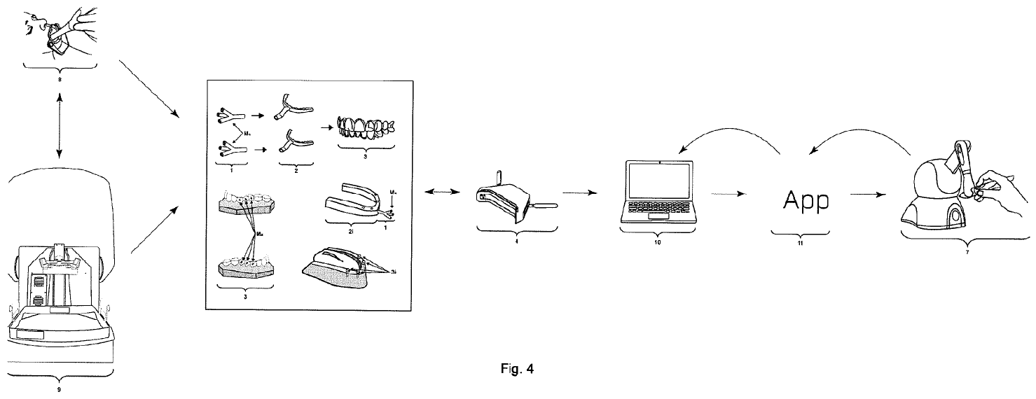

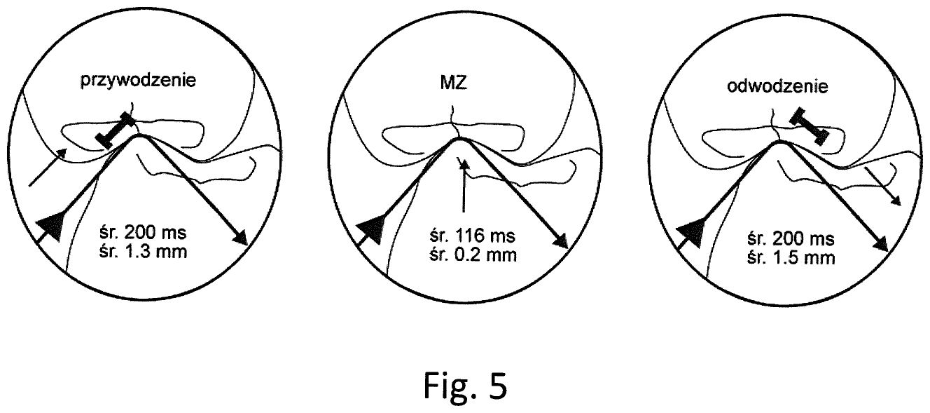

The subject of the invention is presented in one example of embodiment and visualised in the enclosed figures, FIG. 1, FIG. 1a, FIG. 2, FIG. 3, FIG. 4 and FIG. 5.

In FIG. 1, the example presents a model of the positioning pattern, consisting of the external part (1), tipped with three markers (M1) in the form of geometric stickers (optical markers), the internal part (2), individually designed, made, for example, in the 3D printing technology, and rigidly adjacent to the veneer surface of the patient's teeth crowns (3), whereas the internal part (2) and the external part (1) are rigidly joined with each other, for example, by insertion of the end of the external part (1) into the end of the internal part (2).

In FIG. 1a, the example presents a model of the positioning pattern, consisting of the external part (1), tipped with three markers (M1), such as geometric stickers (optical markers), the internal part (2i), individually designed, made, for example, in the 3D printing technology, and rigidly adjacent in the form of clamps/para-occlusion spoons to embrace the supragingival structures, which protrude from the implants are seated on the implants (3i) (for example the impression connectors/transfers for the 3D scanning technique —scan post/scan base), whereas the internal part (2i) and the external part (1) are rigidly joined, for example, by insertion of the end of the external part (1) into the end of the internal part (2i).

In FIG. 2, the example presents the veneer surface of the patient's teeth (3) with markers (M2), placed on it, using the technology of individually designed orthodontic appliances, where the markers are geometric stickers (optical markers).

In FIG. 3, the example illustrates a digital recorder in a monolithic enclosure (4) with an arched recess (5) and two sets of cameras (6). Each camera set contains three cameras, arranged in predefined distances towards one another, whereas each set possesses two monochromatic cameras or colour cameras of a minimum resolution of 2.3 Mpx and a high frame rate of a minimum of 1,000 FPS, with a dedicated optics/lens (preferably with the focus from 35 mm and a diaphragm from f 1.4) and one colour camera of high resolution of a minimum of 12 Mpx and with a low frame rate of a minimum of 25 FPS, whereas the operation of both cameras is synchronised by means of a triggering card(s).

In FIG. 4, the example presents a scheme of actions, aiming at the acquisition and transfer of the information, acquired from the digital image acquisition system, to the haptic manipulator—HM (7) and converting them into vibrations controlled by the tactile sense. In turn, the earlier recorded and defined vibrations from HM (7) can reversely determine/control/impose/prompt such and not another pattern of movement of the upper teeth relative to the lower teeth (by means of dedicated algorithms in the software), individually correlated for every patient and with the amplitude not exceeding 0.2 mm in a set unit of time, in each phase of the chewing cycle, which—in case of construction of new prosthetic embodiments, orthodontic treatment or orthognathic procedures, will protect the periodontium against overloading or the crowns on implants against damage.

In order to position the upper/lower teeth/crowns/supragingival structures seated on implants (3, 3i) relative to one another and to the patient's face (with M2 markers stuck on it at characteristic anatomical points, preferably a minimum of 3 markers at the region of the temporomandibular joints on the right and left side and on the agger nasi), using the markers (M1) by an indirect method with the positioning pattern as in FIG. 1, FIG. 1a, or by a direct method (M2), as in FIG. 2, scanning is needed (8) of the upper/lower crowns/supragingival structures, seated on implants (3, 3i) with markers (M1 and/or M2) and with an optional embodiment of the internal part (2, 2i) of the positioning pattern in the 3D printing technique (9). In turn, a single/exemplary cycle of chewing is recorded by the markers—M1 and/or M2—using the digital recorder in the monolithic enclosure (4). The information from the recorder is then transferred to the computer (10), where, by means of the application (11), are converted into the haptic manipulator operation, such as vibrations controlled by the tactile sense, in particular for the values of the collisions which occur between the pairs of crowns of the upper/lower teeth/crowns/supragingival structures seated on implants (3, 3i), notably larger than 0.2 mm, while smaller than 1.0 mm in a set unit of time.

In FIG. 5, the mean values of the distance and time of the correct phases of the chewing cycle, i.e. of an intact functional envelope, where during the phases, there occur collisions of the pairs of teeth crowns, recorded within the reserved method, which are natural, harmless and tolerated by the chewing organ.

In the preliminary stage of prosthetic construction, a method is selected for the application of markers on the veneer surface of the patient's teeth (3). An indirect mode by means of a positioning pattern, precisely with two independent patterns, one for the veneer surface of the teeth (3) of the lower jaw and one for the veneer surface of the teeth (3) of the upper jaw, whereas each of the patterns consists of an external part (1) with M1 markers, determining the pattern arrangement in the space, and of an internal part (2), matched to the veneer surface of the patient's teeth (3), whereas before measurements, both parts are rigidly joined with each other, or a direct method by a direct application of M2 markers on the teeth (3) of the patient's upper and lower jaw. The direct application of M2 markers on the veneer surface of the patient's teeth (3) is carried out by the method of individually designed orthodontic appliances. Then, the dislocations of the markers (M1, M2) are recorded by the sets of cameras, mounted in a digital recorder (4) with a monolithic enclosure, where the cameras are positioned before the patient's face with M2 markers, stuck on it at characteristic anatomical points (preferably in the region of the temporomandibular joints on the right and left side and on the agger nasi), opposite the mouth or on either side of the face.

In this way of recording, illustrated in the example during the cycle of chewing of interdependent movements of the teeth of the upper and lower jaw, the cameras are connected into 2 systems, one on the right and the other on the left side of the face, whereas each of the systems has 3 cameras, including 2 monochromatic and/or colour cameras of a minimum resolution of 2.3 Mpx and with a high frame rate of a minimum of 1,000 FPS, with a dedicated optics/lens (preferably with the focus from 35 mm and a diaphragm from f 1.4), and one colour camera of a high resolution of a minimum of 12 Mpx and with a low frame rate of a minimum of 25 FPS, that enables the registering of the M1 or M2 markers, fixed by means of clamps/para-occlusive spoons or by themselves to the veneer surface of the upper and lower teeth (3). The operation of the cameras is synchronised in time, thanks to the connections with triggering cards, and is responsible for recording the position/movement of the upper teeth relative to the lower teeth and the patient's face. In addition, it enables the conversion of the markers deformations, recorded by the system of cameras during movement in the software environment, into the number of collisions that occur between the crowns of teeth, natural/implants (3) and M1 and/or M2 markers, which are fixed to the teeth. The acquired information is transferred to a PC (10) and displayed in the digital environment on the screen.

The dislocations of the markers (M1 and/or M2) are recorded by the sets of cameras and converted into digital data which, thanks to the above-mentioned markers, enables the stitching of the images of dentition with the markers into a video with 3D scans of dentition with the markers (M1 and/or M2), obtained by the technique of intra- and extraoral scanning in the application (11) of the computer (10) and then transferred to a haptic manipulator. The collisions, which occur during movement/chewing cycle phases between the crowns of the upper and lower teeth, are converted in the haptic manipulator into vibrations, reflecting these collisions, and recorded by the tactile sense of the user in the frequency from 1 kHz to 4 kHz.

The positioning pattern, mounted in the exemplary embodiment in its individually designed internal part (2), made, for example, in 3D printing technology (9) and rigidly adjacent to the veneer surfaces of the existing crowns of the patient's teeth (3), has a connector, such as a rod, on which the external part is pushed, making a rigid joint, where the external part (1) with M1 markers, such as three stickers/optical markers, placed on its end (1).

The process of data acquisition in the mode applied in the representative example:

1. Two camera systems, (described earlier) triggering cards for synchronic collection and transfer of data (the digital acquisition of image data) from the cameras to the computer.

2. The determination/check/control of the interdependent mobility range of the teeth crowns/supragingival structure(s), seated on implants relative to the bones of the dental processes in both jaws, where the mobility is exerted by stomatological intervention thanks to the records of the markers (M1 and/or M2) dislocation, corresponding to the work of the dentition (the attachment apparatus) from 0.1 mm (for the supragingival structure(s) seated on an implant relative to the implant) to 1.0 mm (preferably above 0.2 mm to 1.0 mm) as an example of disturbed functional work of the teeth relative to one another, i.e. a disturbed functional envelope as a result of the above-mentioned stomatological intervention.

3. The interdependent position and movement of the markers (M1 and/or M2) with the possibility to be entered into the patient's face with M2 markers, stuck on it at characteristic anatomical points (preferably at 3 points in the regions of the temporomandibular joints on the right and left side and on the agger nasi), all the time being monitored by the system of cameras.

4. A minimum interlocking (stitching) of the markers (M1 and/or M2) from the scan of the 3D object with the images of the markers from the video to 0.01 mm preferably to 0.05 mm.

5. The conversion of data from the digital image acquisition into vibrations in the haptic manipulator, controlled by the tactile sense for each phase of movement/chewing cycle with a determination of the harmful values of collisions of the crowns of teeth/supragingival structures seated on implants, causing deformations of the dentition/supragingival structure seated on an implant relative to the implant and exceeding 0.2 mm for the teeth and 0.1 mm for the implants in a set time unit, divided into time intervals, preferably 0.001 second intervals, on the pathway of each phase of the movement/chewing cycle, i.e. on the distance of adduction to intercuspation and on the distance of abduction from intercuspation (200 ms on average).

6. Based on the feedback principle, vibrations may determine/control/impose such and not another pattern of interdependent movements of the crowns of the upper teeth (natural/artificial on implants) relative to the lower teeth in the bone of the dental process of both jaws, not exceeding 0.2 mm in a set unit of time (200 ms on average), divided into time intervals, preferably 0.001 second intervals in each phase of movement/chewing cycle, which, in the case of the construction of new prosthetic embodiments, orthodontic treatment or orthognathic surgery will prevent overloading of the dentition of teeth or protect the crowns on implants from damage.

1. A mode of analysis of a pattern of interdependent movements of the upper jaw and lower jaw teeth by bidirectional synchronising of the digital movement image acquisition technology with haptic technology in a digital analysis of chewing, based on the feedback principle, using Motion Capture technology, based on the position identification of optical markers and recording their movements in camera systems is characterised in that the cameras are arranged into at least two systems, one on the right side and the other on the left side of the mouth/face, or one centrally positioned opposite to the centre line of the face, whereas each of the systems has at least one, preferably three, cameras including at least one (stereoscopic), with two monochromatic or colour cameras of a minimum resolution of 2.3 Mpx and with a high frame rate of a minimum 1,000 FPS (frames per second), with dedicated optics/lens (preferably with the focus from 35 mm and a diaphragm from f. 1.4, and at least one colour camera with a high resolution of a minimum of 12 Mpx and a low frame rate of a minimum 25 FPS, enabling the recording of indirectly fixed markers (M1) with a positioning pattern, or M2 markers—preferably with technology for individually designed dental braces—fixed directly to the veneer surface of the crowns of natural or artificial teeth (the latter installed on the patient's own teeth or fixed on implants) in the upper jaw and, independently, to the veneer surface of the crowns of natural or artificial teeth (the latter installed on the patient's own teeth or fixed on implants) in the lower jaw, (M1 and/or M2), whereas the operation of the cameras is synchronised thanks to their connection with triggering cards, and is responsible for recording of the position/movement of the crowns of the upper teeth (natural/artificial, on implants) with M1 and/or M2 markers relative to the crowns of the lower teeth (natural/artificial, on implants) with M1 and/or M2 markers and of the crowns of the above mentioned teeth (natural/artificial, on implants), upper and/or lower, relative to the bone of the dental process of either jaw, as well as relative to the patient's face with M2 markers stuck on it at characteristic anatomical points (preferably a minimum of 3 markers in the region of the temporomandibular joints on the right and left side and on the agger nasi) thanks to their simultaneous recording in the video film technology and then (thanks to M1 and/or M2 markers) the stitching of 3D scans into the video film, where the 3D scans are the scans of the upper and lower teeth with M1 and/or M2 markers, acquired by the technique of intra- and extraoral scanning, and enables the conversion of dislocations of these M1 and/or M2 markers, recorded by the system of cameras during movement in the course of the examination of the patient's chewing function, followed by the transfer of the data to the software environment, where the digital acquisition of data from the optical markers, M1 and/or M2, is submitted to further analysis, enabling the conversion of these marker dislocations into the number of collisions, which occur between the crowns of the upper and lower teeth (natural/artificial, on implants) with M1 and/or M2 markers, fixed to them, where the vibrations are read by haptic devices, preferably haptic manipulators, enabling the mapping/control these collision-made dislocations, converted into vibrations, by the tactile sense at a frequency between 1 kHz and 4 kHz, enabling in this way the monitoring the course of amplitude changes of the provisional angle of collision, higher/lower than 10 degrees between the pairs of crowns of the upper and lower teeth (natural/artificial, on implants) with M1 and/or M2 markers, in a set unit of time, divided into time intervals, preferably 0.001 second intervals, on the pathway of each phase of movement/chewing cycle, i.e. on the distance of adduction to intercuspation and on the distance of abduction from intercuspation (on average, at a distance of 0.2 mm during the time of 116 ms—for intercuspation, on average, at a distance from 1.3 mm to 1.5 mm in a time of 200 ms—for the adduction phase and the abduction phase, respectively), expressed by the number of vibrations on the haptic manipulator at the moment of contact/collision of these pairs of crowns of the upper and lower teeth (natural/artificial, on implants) with M1 and/or M2 markers in a set unit of time.

2. The mode according to claim 1 characterised in that the positioning of the location of the implants, embedded into the bones of the upper and/or the lower jaw (in case of a total anodontia of the upper and lower jaw) is carried out by means of the supragingival structures, seated on the implants (for example, impression connectors/transfers/transfers for scanning by the 3D technique—scan post/scan base) by the making and rigid fixing of them on an individually designed para-occlusion brackets/spoons, constituting the internal part of the positioning pattern, connected with the ready-made, prefabricated external part of this pattern, protruding from the mouth and tipped with at least three markers, preferably stickers, in order to designate the spatial setting of the positioning pattern and, in this way, of the supragingival structures, seated on the implants, relative to the system of recording cameras, which allows the positioning of the teeth/supragingival structures, seated on the implants, relative to one another within one dental arch, relative to the teeth/supragingival structures, seated on the implants of the opposite dental arch, and for the entrance into the patient's face with M2 markers, stuck on it at characteristic anatomical points (preferably a min. of 3 markers at the region of the temporomandibular joints on the right and left side and on the agger nasi) and/or a video film.

3. The mode according to claim 1, characterised in that in case of the lack of space in dental occlusion or in articulation movements of the crowns of the upper/lower teeth relative to each other, resulting in an inability to record free movement, a method of the markers positioning is used (also for orthodontic treatment procedures) by a direct plotting/positioning of the markers (M2) on the veneer surface of the crowns of the teeth, (natural/artificial, on implants), preferably on the crown of each tooth separately, preferably by the use of the method of individually designed orthodontic appliances.

4. A pattern, positioning the crowns of the teeth, (natural/artificial on implants), characteristic in that it consists of two separable parts, i.e. the external, ready-made, prefabricated part, protruding from the mouth and tipped with at least three markers, preferably optical, so as to designate the spatial setting of the positioning pattern with the markers (M1) relative to the recording cameras, and of the internal part, individually designed and tailored to the veneer surface of the crowns of the patient's teeth (natural/artificial, on implants), whereas the internal part and the external part are rigidly joined with each other.

5. A digital recorder of chewing, characterised in that its monolithic enclosure has the shape of a recess (cavity), such as an arch, made in such a way as to simultaneously record from both sides, right and left, the three-dimensional shape of the face (concave, convex, flat), whereas, at the ends of the arched cavity there are two sets of cameras (or one set, centrally positioned opposite the centre line of the face), recording independently or synchronically the mouth and both sides of the patient's face and the visible markers, M1.
